(19) United States
(12) Patent Application Publication
(10) Pub. No.: US 2011/0177547 A1
(43) Pub. Date: Jul. 21, 2011

(54) Particle Sorting Using Fluid Streams

(75) Inventors: Zheng Xia, Chicago, IL (US); Tania Chakrabarty, Chicago, IL (US); Haojun Fu, Naperville, IL (US); Dan Mueth, Chicago, IL (US)

(73) Assignee: ARRYX, INC., Chicago, IL (US)

(21) Appl. No.: 13/007,176

(22) Filed: Jan. 14, 2011

Related U.S. Application Data
(63) Continuation-in-part of application No. 11/298,565, filed on Dec. 12, 2005, now Pat. No. 7,875,845.
Application No. 11/298,565 is a continuation-in-part of application No. PCT/US10/38332, filed on Jun. 11, 2010.
(60) Provisional application No. 60/634,980, filed on Dec. 13, 2004.
(60) Provisional application No. 60/635,164, filed on Dec. 10, 2004.
(60) Provisional application No. 61/186,825, filed on Jun. 13, 2009.

(51) Int. Cl. C12Q 1/04; G01N 1/20; H05H 3/00; G02B 1/04; B29D 11/00

(52) U.S. Cl. 435/34; 73/863.41; 73/863.61; 250/428; 250/430; 250/251; 428/220; 264/2.7

(57) ABSTRACT

A fluidic device includes an arrangement of channels for introducing a sample containing particles of interest into a processing chamber. The chamber is in fluid communication with collecting channels via low-flow connection channels. Particles in the sample may be observed and diverted from the processing chamber by application of a motive force such as optical trapping into a collection channel. Once in the collection channel, particles can be collected, including by trapping in a porous matrix.

### PRIORITY

This patent application is a Continuation-In-Part of U.S. patent application Ser. No. 11/298,565 entitled AUTOMATED EXTRACTION AND PURIFICATION OF SAMPLES USING OPTICAL TWEEZERS filed Dec. 12, 2005, and claiming priority from U.S. Provisional Patent Application No. 60/634,980 filed Dec. 13, 2004 and U.S. Provisional Patent Application No. 60/635,164 filed Dec. 10, 2004. This patent application is also a Continuation-In-Part of International Patent Application No. PCT/US2010/038332 filed Jun. 11, 2010, entitled PARTICLE SORTING USING FLUID STREAMS, which in turn claims priority from provisional U.S. Patent Application No. 61/186,825 filed Jun. 13, 2009, entitled, PARTICLE SORTING USING FLUID STREAMS. All of the aforementioned patent applications are hereby incorporated by reference in their entirety.

### FIELD OF THE INVENTION

The invention generally relates to sorting small objects including cells and other particles for analytical, forensic, preparative and other purposes.

### BACKGROUND OF THE INVENTION

Genetic material obtained from forensic samples can be used to identify perpetrators of sexual assaults or to exonerate innocent suspects. Purified DNA obtained from sperm cells isolated from forensic samples can be used in subsequent genetic identity testing. The genetic profile of sperm cell DNA can be compared to that of a known suspect or to databases containing genetic information about a large number of convicted felons.

In sexual assault cases, a vaginal or rectal swab, or clothing containing a semen stain, is obtained from the victim for forensic analysis. If sperm cells are present in the sample, DNA from the sperm cells can be isolated and used in genetic identity testing. However, a vaginal swab obtained from a sexual assault victim typically contains relatively few sperm cells and large numbers of epithelial cells from the victim. As a consequence, unless the sperm cells are first separated from other cells in the sample, DNA purified from a forensic sample is susceptible to overwhelming contamination with epithelial cell DNA. Contamination with epithelial cell DNA interferes with the ability to establish a match between the genetic profile of DNA from the sample and that of the suspect or a member of the database. It is therefore desirable to isolate sperm cells from other cells in a forensic sample prior to DNA isolation and analysis.

Techniques currently used to isolate sperm cells from other cells in forensic samples are time consuming and labor intensive, and, as a result, there is currently a backlog of unprocessed samples. Because of this backlog, some jurisdictions have a policy against processing samples unless a suspect has been identified. Consequently, many unprocessed samples are ultimately discarded, and genetic information contained in the sample is never compared with or entered into the national database, which reduces the ability of law enforcement to identify and apprehend repeat sex offenders.

Sperm cells are typically isolated from forensic samples containing epithelial cells by selectively lysing the epithelial cells with Proteinase K and a detergent under nonreducing conditions. Following epithelial cell lysis, intact sperm cells are pelleted by centrifugation and the supernatant, which contains DNA from lysed epithelial cells, is removed. In order to minimize contamination by soluble epithelial cell DNA, the sperm pellet is subjected to repeated washing with an aqueous buffer in an attempt to remove soluble epithelial cell DNA. This process frequently results in the loss of sperm cells.

In addition, prior art methods of analysis may consume most or all of a forensic sample, thereby making retesting impossible or impracticable.

The problem of separating sperm and epithelial is a specific instance of the problem of separating or sorting particles from a fluid, an endeavor that is of interest in various fields of analysis, including cell and biochemical diagnostics.

### SUMMARY OF ILLUSTRATIVE EMBODIMENTS

In accordance with a first embodiment of the present invention, a device includes a processing chamber that is transparent so as to allow the observation of a particle of a first particle type and is permissive to the introduction of a motive force field for the selective diversion of the particle of the first type. The processing chamber has at least a first, second, and third port, and a sample reservoir for holding a sample fluid containing the first particle type and a second particle type. A sample entry flow path connects the sample reservoir to the processing chamber for transporting a sample fluid to the first port and into the processing chamber. A first collection flow path connects a first source of collection fluid to a first collection fluid sink, the first collection flow path having a connection region disposed at a specified distance from the second port of the processing chamber. A first connection channel connects the second port of the processing chamber and the connection region of the first collection flow path. The connection channel is sized so as to create a low-flow region to thereby discourage movement of a particle of the first type from the processing chamber into the first collection flow path in the absence of the motive force field. A sample exit flow path connects the third port of the processing chamber to a sample sink for accepting the sample fluid exiting the processing chamber.

In a related embodiment, the processing chamber allows the observation of a particle of a second type thereby distinguishing it from a particle of the first type for selective diversion under the influence of the force field into the first collection flow path or a second collection flow path connecting a second source of collection fluid to either the first or a second collection fluid sink. The first collection flow path has a connection region disposed at a specified distance from a fourth port of the processing chamber. A second connection channel connects the fourth port of the processing chamber and the connection region of the second collection flow path. The channel is sized so as to create a low-flow region to thereby discourage movement of a particle of the first type from the processing chamber into the first collection flow path in the absence of the motive force.

Various further related embodiments are provided. The device can further include a pumping arrangement adapted to urge fluids through the flow paths. The first or second connection channel can have a length/width ratio of at least 3. The first or second connection channel can have a width of at least 100 micrometers. The sample reservoir can hold a greater volume than the first or second sources of collection fluid. The device can include a trapping material in the first or second collection flow path. The trapping material can have interstices sized to collect particles of the first or second type. One or more of the flow paths can be curved.

In accordance with another embodiment of the present invention there is a system that includes a device as described in one of the above-mentioned embodiments, and an apparatus adapted to identify a particle and to divert the particle into the first or second collection flow paths based on the identification.

Various related embodiments are provided with optional or additional features. For example, the apparatus can use optical tweezing, and optionally, holographic optical tweezing. The system may include an actuation system adapted to urge flow independently through the flow paths. The actuation system may be pneumatic.

In accordance with another embodiment of the invention, there a method sorts particles using a system, as mentioned above. The method includes introducing a sample fluid, identifying a particle, and diverting the particle based on the identification into a collection fluid path.

In accordance with related embodiments, the method also includes further optional or additional steps. The surfaces of the device may be treated with a blocking agent. The first particle type can be sperm and the second particle type can be epithelial cells. The motive force field can be optical tweezing, holographic optical tweezing, magnetic, or electrophoretic. The selective diversion can cause travel along the connection channel.

In accordance with an embodiment of the invention, there is a window for optical interrogation of a fluidic chamber. The window is plastic and is less than 300 micrometers in thickness. The window has a flatness of 100-200 micrometers in height per millimeter of length. The window may be manufactured by a process that includes molding or embossing the window, preferably with a diamond-polished tool.

In accordance with an embodiment of the invention, a method for observing a particle in a fluidic device includes optically interrogating the particle via a window according to and optionally applying a holographic aberration correction.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1a schematically shows a microfluidic device in accordance with in embodiment of the invention;

FIG. 1b shows the device of FIG. 1a in the context of a forensic application and further having valving/pumping arrangement;

FIG. 2 is a schematic diagram of a microfluidic cartridge in accordance with an embodiment of the invention;

FIG. 3 is a photograph of a microfluidic cartridge constructed from multiple layers;

FIG. 4 is a photomicrograph of a processing chamber window in accordance with an embodiment of the invention;

FIG. 5 is a schematic plan view of a microfluidic cartridge constructed from multiple layers;

FIG. 6 is an exploded perspective schematic view of the cartridge of FIG. 5;

FIG. 7 is an exploded perspective schematic view of an alternate construction of a fluidic cartridge;

FIG. 8 schematically illustrates a process for constructing a multilayer fluidic cartridge;

FIGS. 9a-c schematically show a sequence of fluidic operation of a cartridge in accordance with an embodiment of the invention;

FIG. 10 shows a schematic plan view of a microfluidic device that includes branched channels in accordance with an embodiment of the invention;

FIG. 11 shows a device in accordance with the embodiment of FIG. 10 having a space-saving design;

FIG. 12a-j schematically show a sequence of fluidic operations in accordance with an embodiment of the invention;

FIG. 13 shows a microfluidic chip in connection with the embodiment of FIGS. 12a-j;

FIG. 14a shows a schematic plan view of a valve/pump in a closed position;

FIG. 14b shows a sectional schematic view of the valve/pump of FIG. 14a in a closed position;

FIG. 14c shows a schematic plan view of the valve/pump of FIGS. 14a-b in an open position; and

FIG. 14d shows a sectional schematic view of the valve/pump of FIG. 14a-c in an open position.

### DESCRIPTION OF ILLUSTRATIVE EMBODIMENTS

Embodiments of the present invention allow for rapid separation or sorting of precise numbers of particles in a sample. In illustrative embodiments, particles are identified and separated from a stream of sample liquid through the application of a motive force field (e.g., optical tweezing). (As used herein, a “particle” refers to any object capable of being separated or sorted under the influence of the field in connection with embodiments the invention, regardless of shape of size. For example, cells, polystyrene beads and hair fibers are “particles”). Liquid sample, potentially containing particles of interest, is fed into a processing chamber from a reservoir. While in the reservoir, particles of a specified type are identified and diverted under influence of the motive force field into a collection flow path or channel for collection and downstream analysis or use of the collected particles. The processing chamber and collection flow path are separated by a low-flow connection channel that acts to prevent unwanted crosstalk of materials between the sample stream and a collection stream flowing in the collection flow path. Accordingly, a more pure sample of collected particles is produced. Additional collection flow paths and connection channels may be used to separate multiple particle types. Details of illustrative embodiments are discussed below, with reference to separating sperm from epithelial cells for forensic application (i.e., analysis of a rape kit). However, the devices and methods may be used for other cell and non-cell particle separation. The collected particles may also be used for non-analytical purposes also. For example, red blood cells and platelets could be separated and either administered to a patient in need.

Embodiments of the invention may utilize instrumentation and analytical methods described in U.S. Published Patent Application No. 20090075826 to Chakrabarty, which is hereby incorporated herein in their entirety.

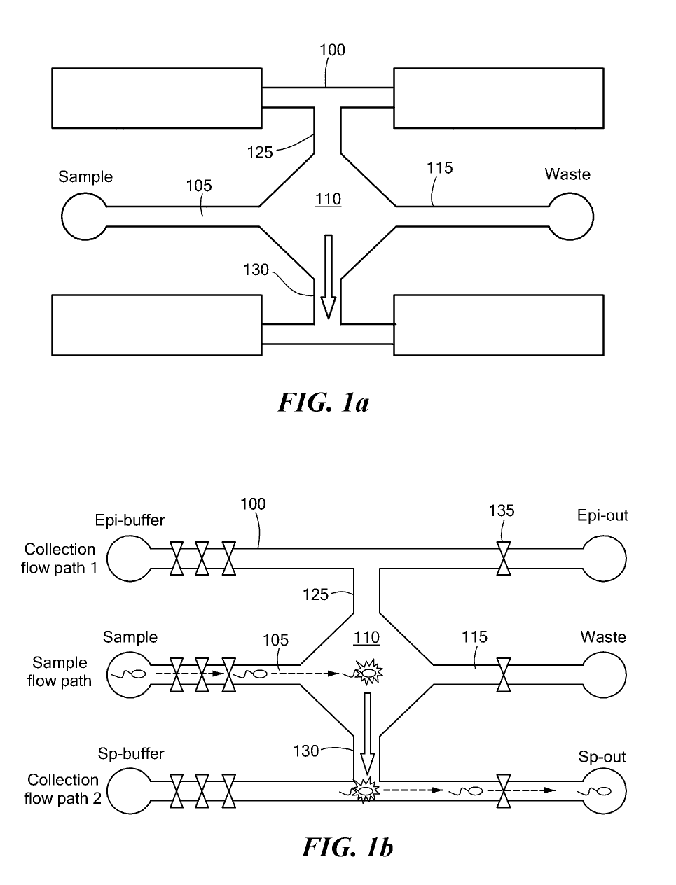

FIGS. 1a-b shows a schematic layout of a microfluidic device 100 in accordance with an embodiment of the invention. FIG. 1a shows the general case and FIG. 1b shows a more specific example in the context of sorting sperm from epithelial cells using a valving/pumping arrangement. A sample flow path has two branches: a sample entry flow path 105 leading to a processing chamber 110 and an exit flow path 115 exiting from the processing chamber 110. Two collection channels are connected to the processing chamber via connection channels 125, 130. The processing chamber is shown as having a square shape, but may also can be configured in other shapes including rounded shapes.

In use, a sample fluid containing multiple particle types (e.g. sperm from a rapist and vaginal epithelial cells from a victim in an aqueous buffer), is introduced (e.g., by pumping) into a reservoir upstream of the sample inlet channel of the sample flow path. The processing chamber 110 is transparent to interrogating radiation (e.g., visible light) for imaging the chamber contents and identifying particles. For example, the sperm and epithelial cells may be identified using a microscope focused on the sample through an optical window of the chamber. The microscopy and analysis, including the identification, may be automated. Once a particle with desired characteristics (e.g., the sperm shown entering the chamber) is identified, a motive force field may be applied to move the particle to one of the collection flow paths.

In embodiment, the motive force field is optical trapping. Multiple optical traps may also be used. Holographic Optical Trapping (HOT) may be used to create the one or more optical traps. A suitable system for performing moving one or more particles using HOT may employ or be based on the BioRyx 200, sold by the applicant, Arryx, Inc. of Chicago, Il. Other embodiments of the invention use other motive forces; for example, magnetic forces to move magnetic particles, or electrophoretic forces to move charged particles. For example, using HOT, 4 sperm may be diverted simultaneously at a velocity of about 100 micrometers per second for collection of about 400 cells in 30 minutes.

The processing chamber 110 is connected to collection flow paths via connection channels 125, 130. Each connection channel is wide enough to allow travel of a particle from the processing chamber under the influence of the motive force field and may have an aspect ratio (length/width) that results in a low-flow region in the connection channel. As a result of the channel length and low-flow properties, diffusion, convection, or other transfer of materials through the connection channel that is not due to the motive force-field is greatly reduced. As a result, there may be limited or no detectable crosstalk between the channels.

Optionally, crosstalk can be further reduced by closing valves in channels that are unused in a given fluidic operation. For example, valves 135 in the collection channels may be closed while filling the processing chamber. In this case, reduced crosstalk occurs because aqueous solutions in the collection and connecting channels are highly incompressible. Figures and corresponding description below show how to use pumping/valving sequences to reduce crosstalk.

In specific embodiments, the connection channel aspect ratio may be from about 3 to about 20. The length of the channels may be so short as to be merely a constriction, but is preferably longer to better prevent unwanted particle communication (e.g., 100 to 1500 micrometers). In a particular illustrative embodiment, the length is 600 micrometers and the width is 150 micrometers, for an aspect ratio of 4. Thus, at a velocity of 100 micrometers per second, in this embodiment a particle would traverse the connection channel in 6 seconds. Using HOT to produce 4 optical traps would speed the particle flux through the channel to one particle every 1.5 seconds. Although the specific details of the number of optical traps and rate may vary, these parameters are generally chosen so as not to destroy the desired particle due to excess optical power. Not withstanding the above, it is possible to use laser power to deliberately inactivate unwanted particles.

Cell-depleted or excess sample may be pumped from the processing chamber to a waste sink. Likewise, each collection flow path may lead to an outlet for recovery of the particle-enriched collection fluid.

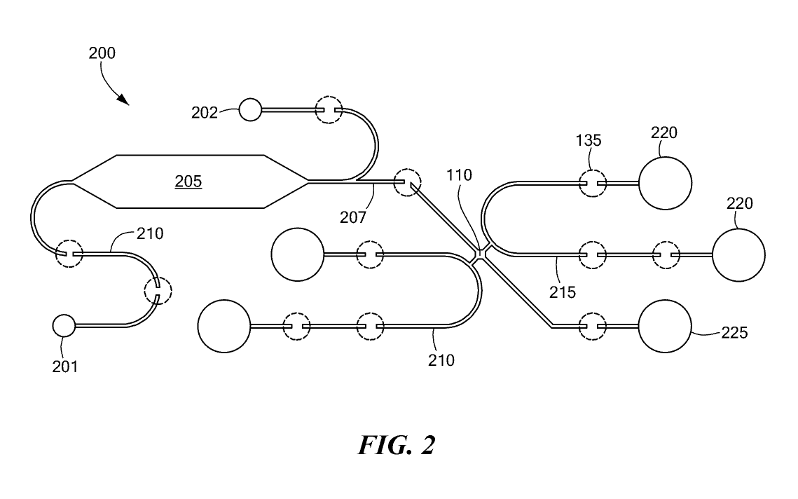

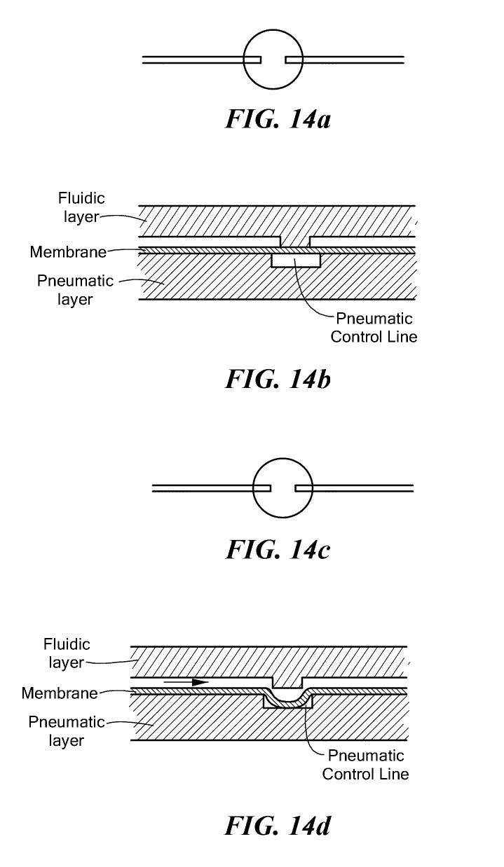

FIG. 2 is a schematic diagram of a microfluidic cartridge (“chip”) 200 in accordance with an embodiment, having a sample fluid reservoir 205 upstream of a sample flow path. The reservoir is loadable via a sample loading flow path 210, which may be actively pumped so that sample is introduced at sample inlet 201 and excess sample or air is expelled at sample outlet 202, thereby filling the reservoir 205. Pumping along the various flow paths may be accomplished using pneumatically pump/valves. Such pumping arrangements are disclosed in US Published Patent Application No. 2007/0166199, hereby incorporated by reference in relevant part. FIGS. 14 a-d, discussed below, also show an example of a pump/valve. Other fluidic pumping arrangements known in the art may also be used, including syringe pumps, electro-osmosis, gravity-feed, etc. One or more of the flow paths may be curved to save space on the chip. Note that the fluid may flow in either orientation through the side channels. However, specific inlet and outlet structures may be included that are best use with, or require, flow in a particular direction.

Sample fluid flows from the sample reservoir 205 through the processing chamber 110. Particles may be diverted into alternate collection channels 210, 215, which terminate in inlets/outlets 220. Processing sample liquid flows to a sample sink 225.

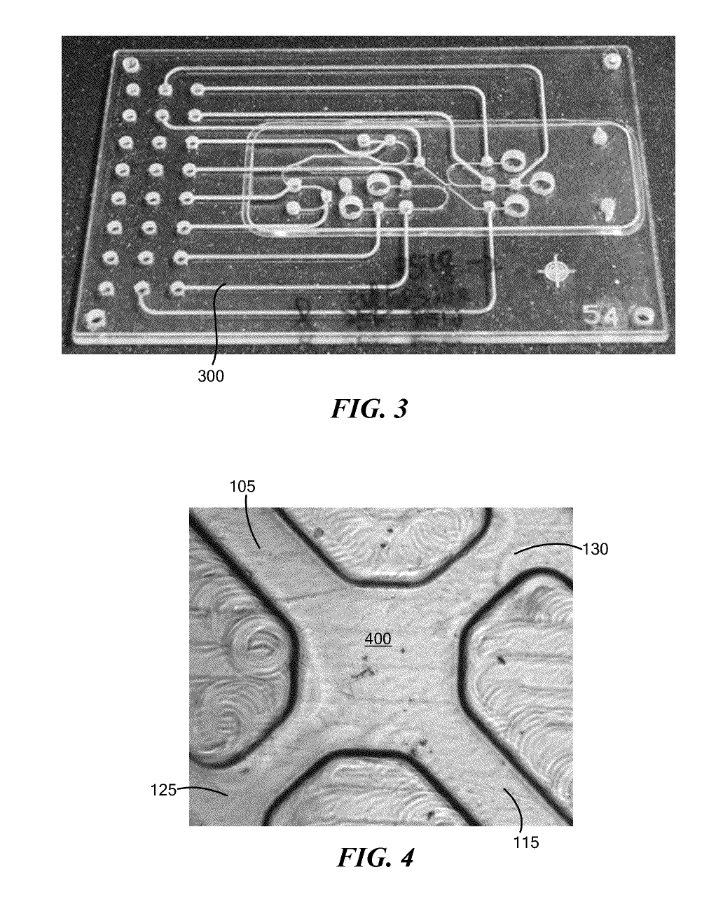

FIG. 3 is a multilayer thermoplastic microfluidic chip 200 in accordance with the embodiment of FIG. 2. The channels 300 of the upper layer are pneumatic channels for actuation of pumping and valving as needed. Theses channels terminate in ports 310 for connection to a pneumatic actuating manifold (not shown). The chip 200 is approximately the size of a credit card (which is known to be about 86×54 mm).

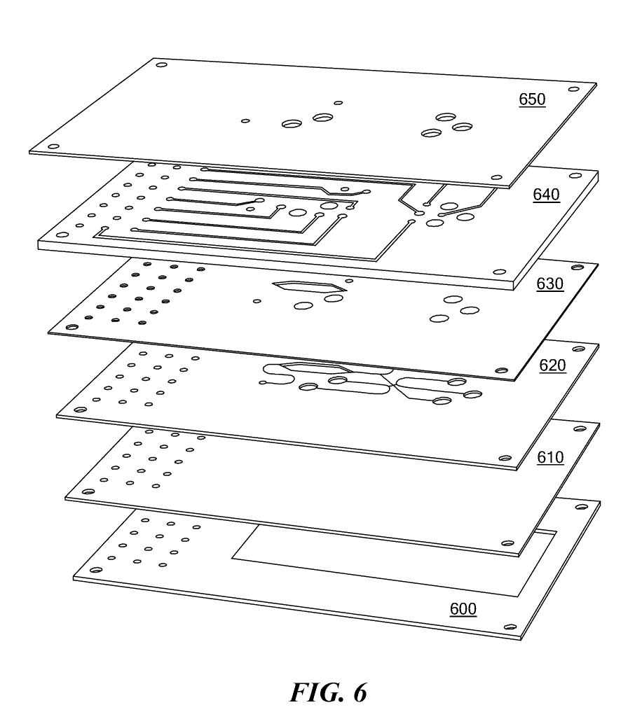

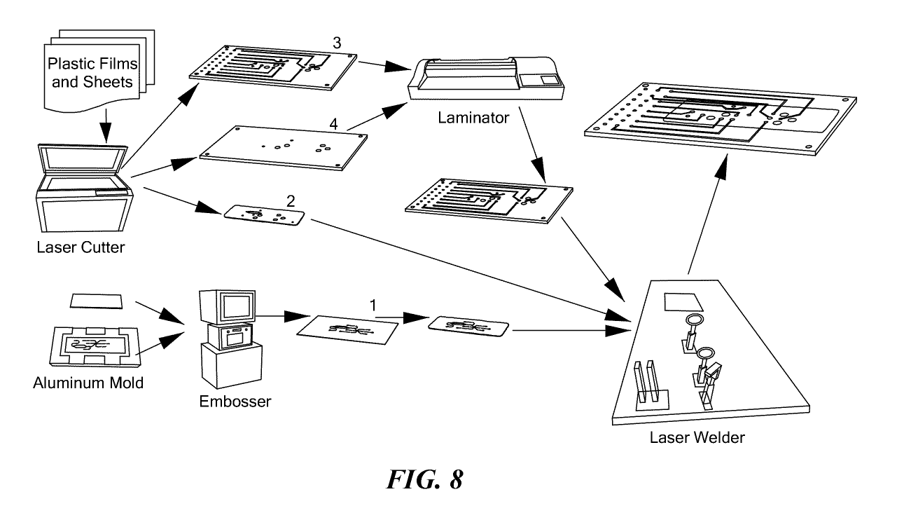

FIG. 4 shows a microphotograph of the processing chamber of FIGS. 1-3. The chamber includes a flat, smooth, and thin plastic window 400 of processing chamber 110, suitable for optical viewing and diversion of particles using HOT. The window 400 may be molded or embossed in to the device using a suitable flat and smooth die. The die may be sized to produce a window that is less than 300 micrometers thick, thereby giving high transparency and adequate strength. The optical properties of plastic are typically poorer than for glass. For example, using plastic was found to give about a 30% reduction in performance for HOT trapping. However, it was found that aberrations could be corrected through holographic aberration correction (encoding the reverse of the aberration and subtracting the differential wavefront). In addition, the plastic window may be made as smooth as possible. For example, the mold used to make the window may be diamond polished. The gross flatness of the window may be 100-200 microns in height per millimeter of length, thereby allowing an optical instrument to maintain focus during travel through the chamber. A similar flatness may be achieved in the connection channels 125, 130. As described below in FIGS. 6 and 8, the chip may be assembled from multiple layers. To preserved the plastic window with as little aberration as possible, the window may be kept cool (below of deformation temperature characteristic of the plastic used). This may be accomplished by precisely laser-welding the plastic parts together, rather than heating globally.

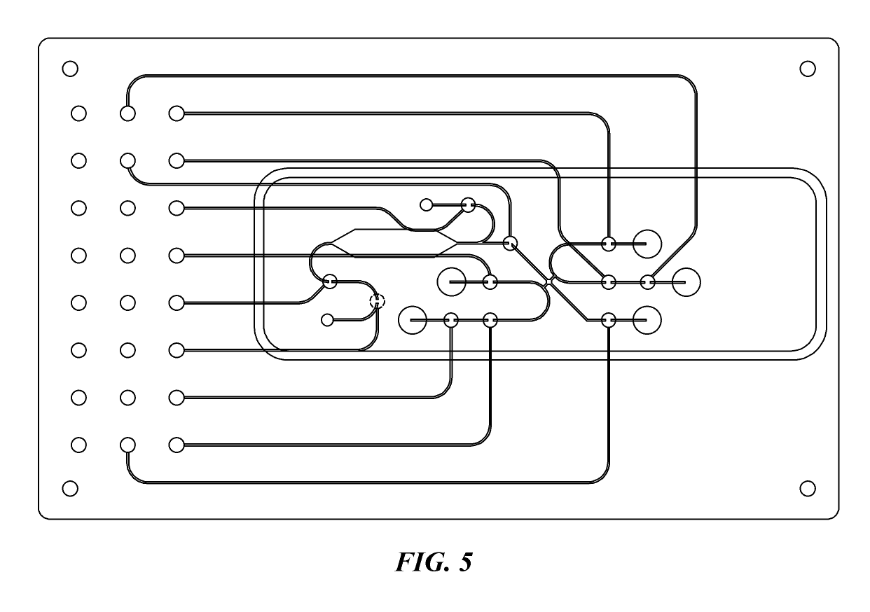

FIG. 5 shows a schematic view of the microfluidic chip 200 of FIG. 2 assembled with a pneumatic control structure as in FIG. 3.

FIG. 6 shows an exploded schematic view of a microfluidic chip 200 assembled from 5 layers, in accordance with an embodiment of the invention. The chip 200 includes a gasket layer 600, a bottom layer 610, a fluidic layer 630, a membrane layer 640, a pneumatic layer 650, and a top layer 660.

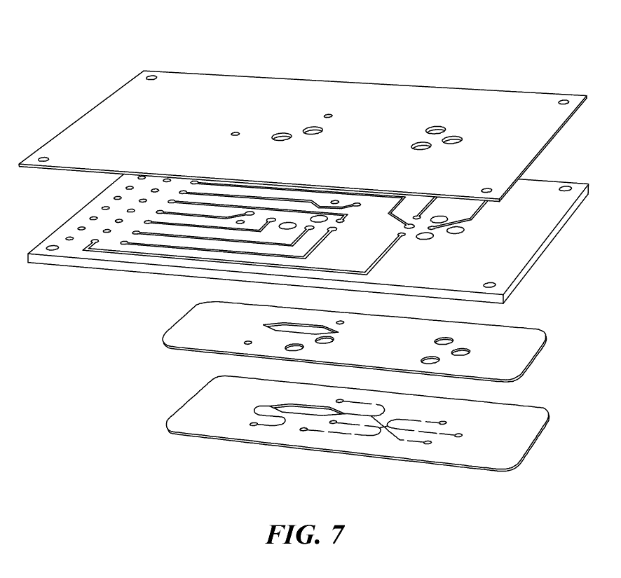

FIG. 7 shows an exploded schematic view of a microfluidic chip assembled from only 4 layers, in accordance with an embodiment of the invention. As described below in connection with FIGS. 14a-d, the flexible or elastic membrane layer of the embodiments of FIGS. 6 and 7 serves to open and close valving structures under the influence of pneumatic actuation, and combing actuation of different pump/valves induces liquid flow in the chip.

FIG. 8 shows a work flow for producing a flow chip in accordance with the embodiment of FIG. 6. The chip 200 may be produced by embossing, laminating, and laser-welding the sheets together. Other techniques for producing chip 200 are well known in the art of microfluidics.

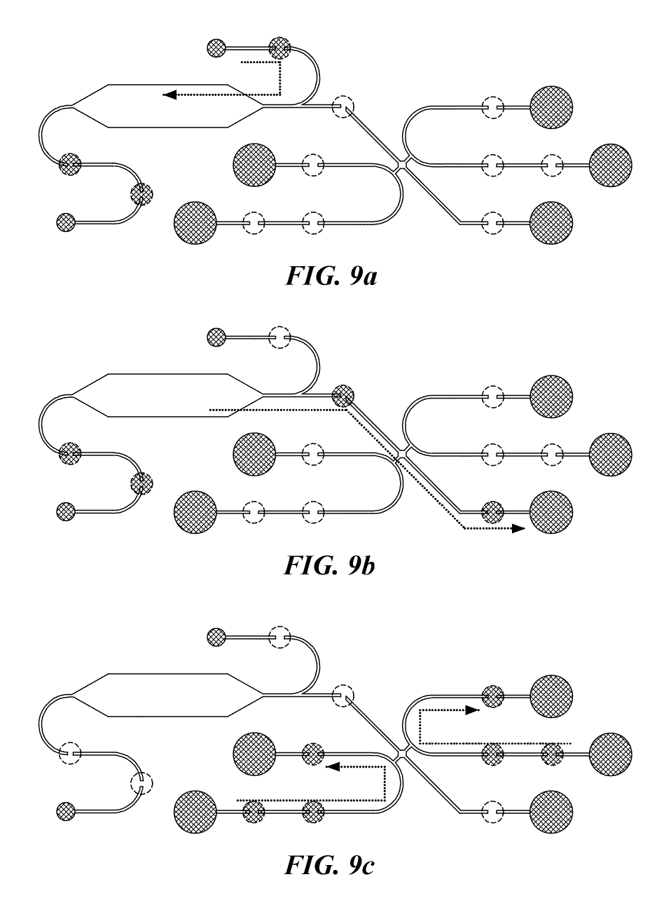

FIGS. 9a-c schematically shows a sequence of major fluidic operations on the chip, in which dashed circles indicate valve/pumps in a closed state and filled circles indicate valve/pumps being actuated. Dashed lines indicate direction of flow. Sample is fed into the reservoir 205; then the pumping valves 135 are used to urge sample fluid from the reservoir through the processing chamber 110 (FIG. 9a). Some of the fluid may go to the waste sink 225 to ensure filling of the processing chamber 110 (FIG. 9b). Particles are then identified and transported to one of the collection channels 210 or 215, and are transported to a collection chamber for future use. A more detailed sequence of fluidic operations is given below.

Particles may be stored on the chip, dispensed or extracted as an aqueous solution. In an embodiment, a collection medium (e.g., porous or fibrous material) is included in a downstream region of a collection channel 210 and/or 215. The material may have an affinity for the particle to be collected. When porous, the interstices of the collection medium may be sized to collect particles of a particular type. For example, cells may be collected in cotton fibers, cotton wool, glass wool, a cotton filter, or a non-cotton filter. The medium may be then removed and optionally dried, fixed or otherwise preserved for future use. Alternately, the sample may be dried or otherwise preserved in the material while on the chip and stored on the chip for later use. Later use may entail extraction and processing of DNA from the medium. These techniques may be used to store sorted sperm and epithelial cells, or to store unused sample fluid for later use.

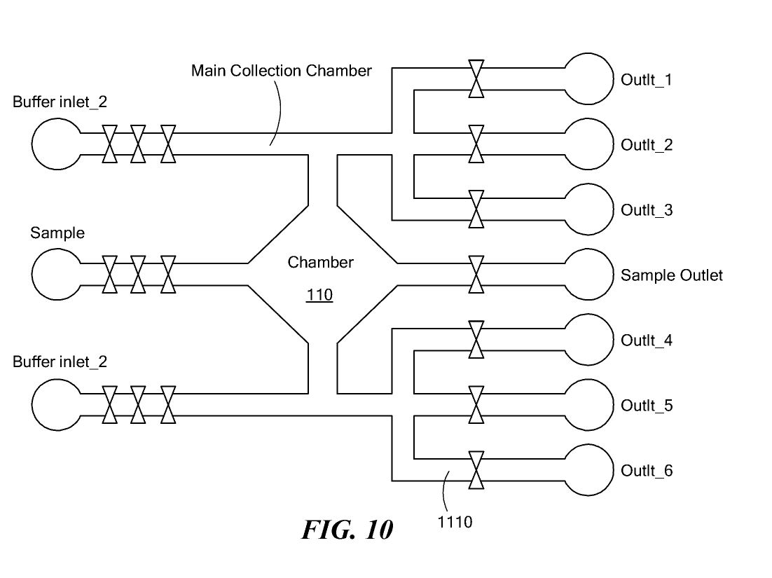

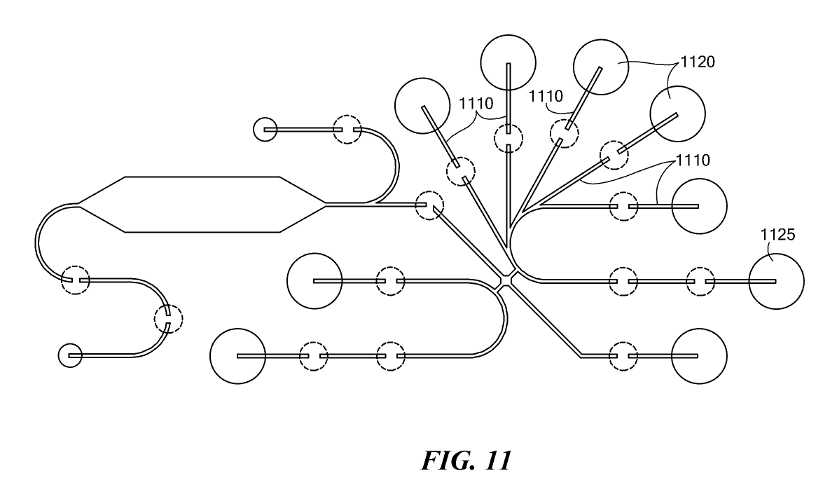

FIG. 10 is a schematic diagram showing an embodiment of the invention in which one or more sample collection flow paths are divided into two or more collection branches 1110 downstream of the corresponding connection channels. Each branch is individually valved and actuable so that collection fluid may be switched for collection in the different branches. In the example illustrated, six types of particles may be collected in the six outlets 1120. (Item 1125 is an inlet) In operation, two different particle types may be sorted into the opposing main collection channels at one time. The chip may then switch modes to select one or more different particle types and to divert the different particle type to a different outlet branch 1110 by opening the valve to that branch and closing the valves to neighboring branches of the same collection channel. Likewise, a different particle mode may be selected for sorting to an outlet 1120 of the opposing collection channel. FIG. 11 shows a space-saving chip layout for the embodiment of FIG. 10. Space is saved by using curved channels.

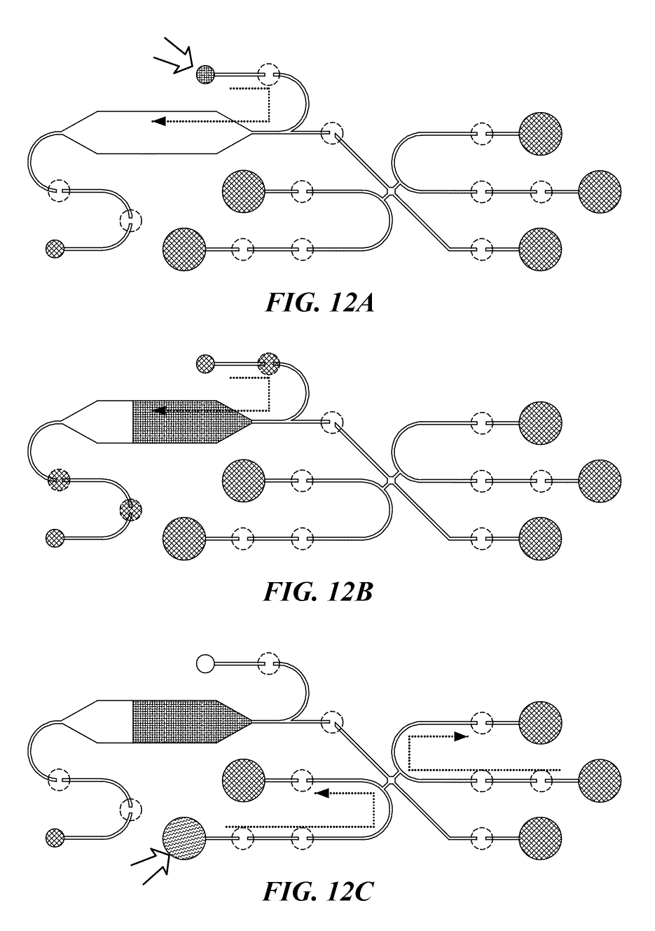

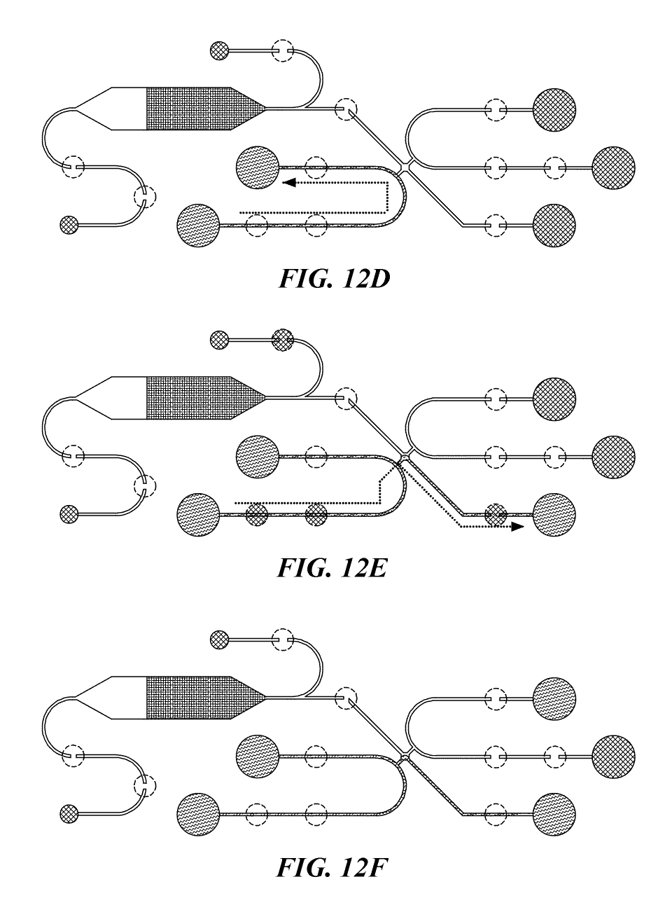

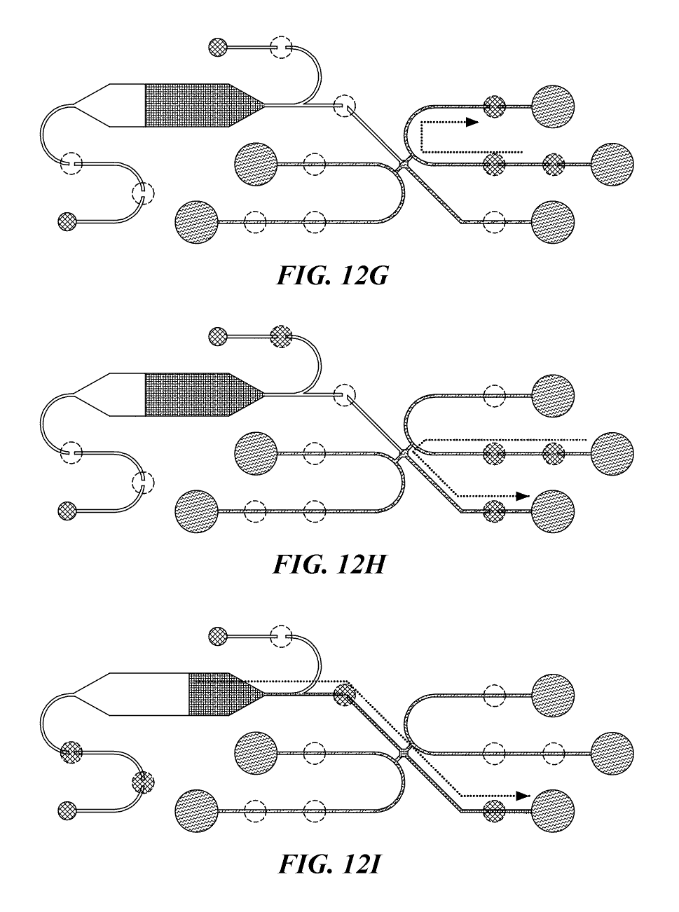

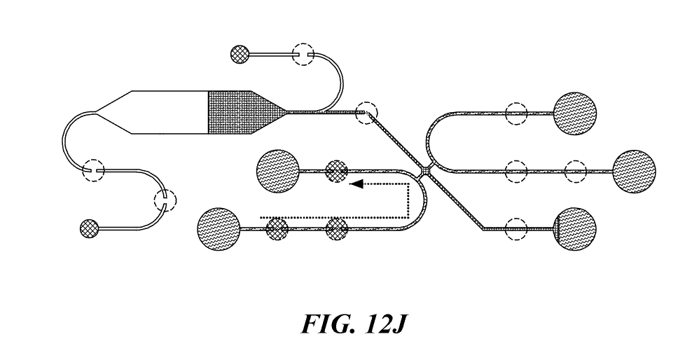

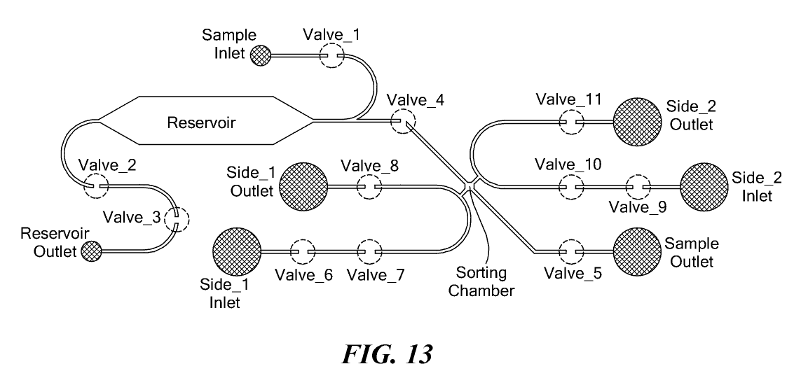

A sequence of fluidic operations in accordance with an illustrative embodiment of the invention is shown schematically in FIGS. 12A-12J. In the explanation below, note that the valve may also serve a pumping function. The description of the steps below uses nomenclature specified in FIG. 13.

Step 1: Load sample solution in the sample inlet well. Dashed circle indicates valve is in a closed state. The device state is shown schematically in FIG. 12A.

Step 2: Pump sample solution from sample inlet to reservoir. Solid circle indicates valve is in operation. The device state is shown schematically in FIG. 12B.

Step 3: Load buffer solution in the inlet well of side_{—}1 channel. The device state is shown schematically in FIG. 12C.

Step 4: Pump buffer solution from side_{—}1 inlet to side_{—}1 outlet well so as to fill the side_{—}1 channel. The device state is shown schematically in FIG. 12D.

Step 5: Pump buffer solution from side_{—}1 inlet to sample outlet, so as to fill the channel connecting side_{—}1 channel to chamber. The device state is shown schematically in FIG. 12E.

Step 6: Load buffer solution in the inlet well of the other side channel. The device state is shown schematically in FIG. 12F.

Step 7: Pump buffer solution from side_{—}2 inlet to side_{—}2 outlet well so as to fill the side_{—}2 channel. The device state is shown schematically in FIG. 12G.

Step 8: Pump buffer solution from side_{—}2 inlet to sample outlet well, so as to fill the channel connecting side_{—}2 channel to chamber. The device state is shown schematically in FIG. 12H.

Step 9: Pump sample solution from reservoir to sample outlet, so as to load sample solution into the sorting (processing) chamber. The device state is shown schematically in FIG. 12I.

Step 10: Once target cells are trapped and moved through the connecting channel into side_{—}1 channel, pump buffer solution in the side_{—}1 channel so as to drive the target cells into the side_{—}1 outlet well. The device state is shown schematically in FIG. 12J.

Step 9 and step 10 are repeated until desired number of cells are sorted and collected from the outlet well.

TABLE 1

| Operation | Valves in | |
|---|---|---|
| step | operation | Operation description |
| 1 | | Load sample solution in the sample inlet well |
| 2 | 1, 2, 3 | Pump sample solution from sample inlet to |
| | | reservoir |
| 3 | | Load buffer solution in the inlet well of side_1 |
| | | channel |
| 4 | 6, 7, 8 | Pump buffer solution from side_1 inlet to side_1 |
| | | outlet well so as to fill the side_1 channel |
| 5 | 6, 7, 5 | Pump buffer solution from side_1 inlet to sample |
| | | outlet, so as to fill the channel connecting side_1 |
| | | channel to chamber |
| 6 | | Load buffer solution in the inlet well of the other |
| | | side channel |
| 7 | 9, 10, 11 | Pump buffer solution from side_2 inlet to side_2 |
| | | outlet well so as to fill the side_2 channel |
| 8 | 9, 10, 5 | Pump buffer solution from side_2 inlet to sample |
| | | outlet well, so as to fill the channel connecting |
| | | side_2 channel to chamber |
| 9 | 3, 2, 4, 5 | Pump sample solution from reservoir to sample |
| | | outlet, so as to load sample solution into the |
| | | sorting chamber |
| 10 | 6, 7, 8 | Once target cells are trapped and moved through |
| | | the connecting channel into side_1 channel, |
| | | pump buffer solution in the side_1 channel so as |
| | | to drive the target cells into the side_1 outlet well |

FIGS. 14a-d illustrate a normally-closed pumping valve arrangement that can be used in connection with the above-described embodiments. In the closed position of FIG. 14a-b, the membrane seals the fluidic channel so that liquid cannot flow. A positive pneumatic pressure (e.g., 10-20 psi) may be applied to the membrane via a pneumatic control line of the pneumatic layer to ensure proper seating and sealing. When a negative pressure (e.g., a vacuum of −10 psi) is applied, the membrane is pulled into a pneumatic layer, thus opening the valve and permitting flow (e.g., in the direction of the arrow), as in FIG. 14c-d. Because the opening of the valve may draw liquid into the newly exposed fluidic volume (a “pumping chamber”), subsequent closing of the valve will create a pumping action.

In use, non-specific absorption to walls of the channels, membranes of other wetted surfaces may adversely impact performance of the chip. Adequate blocking can be especially important for movement of particles from the sample fluid to the collection fluid under influence of the motive force field; otherwise, particles may stick to the surfaces, thereby preventing transfer. This may also lead to occlusion of the connecting channel. This problem may be combated by treating the wetted surfaces with one or more covalent or noncovalently acting blocking agents during manufacture, after manufacture but before use, or during use by including blocking agents in the sample fluid and/or collection fluids. Suitable blocking agents may include bovine serum albumin (BSA), casein, and other blockers known in the art. Combinations of blocking agents may also be used.

In alternative embodiments, the disclosed methods for particle sorting and/or analysis may be implemented as a computer program product for use with a computer system. Such implementations may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems.

Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

Although the above discussion discloses various exemplary embodiments of the invention, it should be apparent that those skilled in the art can make various modifications that will achieve some of the advantages of the invention without departing from the true scope of the invention.

What is claimed is:

1. A device comprising:
a processing chamber, transparent so as to allow the observation of a particle of a first particle type and permissive to the introduction of a motive force field for the selective diversion of the particle of the first type, the processing chamber having at least a first, second, and third port;
a sample reservoir for holding a sample fluid containing the first particle type and a second particle type;
a sample entry flow path connecting the sample reservoir to the processing chamber for transporting a sample fluid to the first port and into the processing chamber;
a first collection flow path connecting a first source of collection fluid to a first collection fluid sink, the first collection flow path having a connection region disposed at a specified distance from the second port of the processing chamber;
a first connection channel connecting the second port of the processing chamber and the connection region of the first collection flow path, the channel sized so as to create a low-flow region to thereby discourage movement of a particle of the first type from the processing chamber into the first collection flow path in the absence of the motive force field;
a sample exit flow path connecting the third port of the processing chamber to a sample sink for accepting the sample fluid exiting the processing chamber.

2. A device according to claim 1, wherein the processing chamber allows the observation of a particle of a second type thereby distinguishing it from a particle of the first type for selective diversion under the influence of the force field into the first collection flow path or a second collection flow path connecting a second source of collection fluid to either the first or a second collection fluid sink, the first collection flow path having a connection region disposed at a specified distance from a fourth port of the processing chamber; and
a second connection channel connecting the fourth port of the processing chamber and the connection region of the second collection flow path, the channel sized so as to create a low-flow region to thereby discourage movement of a particle of the first type from the processing chamber into the first collection flow path in the absence of the motive force.

3. A device according to claim 1 or 2, further comprising a pumping arrangement adapted to urge fluids through the flow paths.

4. A device according to claim 1 or 2, wherein the first or second connection channel has a length/width ratio of at least 3.

5. A device according to any of the preceding claims, wherein the first or second connection channel has a width of at least 100 micrometers.

6. A device according to any of the preceding claims, wherein the sample reservoir holds a greater volume than the first or second sources of collection fluid.

7. A device according to any of the preceding claims, further comprising a trapping material in the first or second collection flow path the trapping material having interstices sized to collect particles of the first or second type.

8. A device according to any of the preceding claims, wherein at least one of the flow paths is curved.

9. A system comprising, a device according to any of the preceding claims, and further comprising an apparatus adapted to identify a particle and to divert the particle into the first or second collection flow paths based on the identification.

10. A system according to claim 9, wherein the apparatus uses optical tweezing, and optionally, holographic optical tweezing.

11. A system according to claim 9 or 10, further comprising an actuation system, optionally a pneumatic actuation system adapted to urge flow independently through the flow paths.

12. A method for sorting particles comprising proving a system according to claim 10, introducing a sample fluid, identifying a particle, and diverting the particle based on the identification into a collection fluid path.

13. A method according to claim 12, further comprising treating surfaces of the device with a blocking agent.

14. A method according to claim 12, wherein the first particle type is sperm and the second particle type is epithelial cell.

15. A method according to claim 12, wherein the motive force field is one of optical tweezing, holographic optical tweezing, magnetic, or electrophoretic.

16. A method according to claim 12, wherein the selective diversion causes travel along the connection channel.

17. A microfluidic device comprising:
means for introducing a sample fluid;
means for identifying a particle in the sample fluid;
means for diverting the particle based on the identification,
means for collection of the particle.

18. A window for optical interrogation of a fluidic chamber comprising a plastic and being less than 300 micrometers in thickness and having a flatness of 100-200 micrometers in height per millimeter of length.

19. A method for manufacturing a window according to claim 18, comprising molding or embossing the window, preferably with a diamond-polished tool.

20. A method for observing a particle in a fluidic device comprising optically interrogating the particle via a window according to claim 18 and optionally applying a holographic aberration correction.
